(19) United States
(12) Patent Application Publication
(10) Pub. No.: US 2018/0264282 A1
(43) Pub. Date: Sep. 20, 2018

(54) Specially Designed Kit To Aid Photo-Biologic Eradication Of Staphylococcus Aureus And MRSA In The Nares

(71) Applicant: Nomir Medical Technologies, Inc., Cedarhurst, NY (US)

(72) Inventor: Eric Bornstein, Woodmere, NY (US)

(21) Appl. No.: 15/922,449

(22) Filed: Mar. 15, 2018

Related U.S. Application Data
(60) Provisional application No. 62/472,443, filed on Mar. 16, 2017.

(51) Int. Cl. A61N 5/06; A61K 31/351

(52) U.S. Cl. CPC A61N 5/0601; A61K 31/351; A61N 2005/0659; A61N 2005/0635; A61N 2005/0607

(57) ABSTRACT

Nasal colonisation with pathogenic bacteria continues to present challenges for patients undergoing surgical procedures, and the physicans that treat them. Many physicians and scientists have discussed different ways to improve nasal decolonization in the last 100 years. Various embodiments include a medical kit composed of items specifically tailored to assist in the photo-biologic eradication of bacteria in the human nares.

### RELATED APPLICATIONS

This application claims the benefit of priority to U.S. Provisional Patent Application No. 62/472,443 entitled “Specially Designed Kit To Aid Photo-Biologic Eradication Of Staphylococcus Aureus And MRSA In The Nares” filed Mar. 16, 2017, the entire contents of which are hereby incorporated by reference for all purposes.

### BACKGROUND

Since the advent of sulfa drugs and penicillin, exploitation of significant quantities of antimicrobial agents of all kinds across the planet has created a potent environment for the materialization and spread of resistant contaminants and pathogens. Certain resistant contaminants take on an extraordinary epidemiological significance, because of their predominance in hospitals and the general environment. Widespread use of antibiotics not only prompts generation of resistant bacteria; such as, for example, methicillin-resistant Staphylococcus aureus (MRSA) and vancomycin-resistant Enterococci (VRE); but also creates favorable conditions for infection with the fungal organisms (mycosis). Given the increasing world's population and the prevalence of drug resistant bacteria and fungi, the rise in incidence of bacterial or fungal infections is anticipated to continue unabated for the foreseeable future.

Staphylococcus aureus (S. aureus) is a gram-positive cocci that is in the human commensal microbiome. As a potential pathogen, S. aureus colonizes the skin and various mucosal surfaces in several parts of the body, including the nasal cavity (nares), of roughly 30% of the human population.

Currently, available therapies for bacterial infections include administration of antibacterial therapeutics or, in some instances, application of surgical debridement of the infected area. Because antibacterial therapies alone are rarely curative, especially in view of newly emergent drug resistant pathogens and the extreme morbidity of highly disfiguring surgical therapies, it has been imperative to develop new strategies to treat or prevent microbial infections.

### SUMMARY

Systems, methods, and devices of various embodiments may enable photo-biologic nasal decolonization of bacteria within a nasal passage of a subject using a therapeutic light delivery system. The therapeutic light delivery system in various embodiments may include an optical radiation generation device, a controller, a delivery assembly, and a medical procedure kit. In some embodiments, the delivery assembly may include an optical fiber adapted for transmitting near-infrared (NIR) radiation to a treatment site within the nasal passage, and a diffusion device. In some embodiments, the medical procedure kit may include at least one piece for use in removing debris and softening sebum from the openings to hair follicles and sebaceous glands in the nasal passage. In some embodiments, the medical procedure kit may also include at least one disposable sleeve for the diffusion device, and at least one topical antibiotic for dispensing into the nasal passage.

In some embodiments, the at least one disposable sleeve may be made of an optically-transmissive medical grade plastic that is transparent to one or multiple NIR wavelengths. In some embodiments, the at least one disposable sleeve may be configured with a parabolic or spherical reflective inner surface at an apex. In some embodiments, the parabolic or spherical reflective inner surface may be configured to produce micro-scattering of light. In some embodiments, the parabolic or spherical reflective inner surface may be configured to reflect excess NIR light traveling away from the treatment site back to the nasal passage. In some embodiments, the apex of the at least one disposable sleeve may be positioned such that a diffuse spread of the excess light back towards the nasal passage is produced. In some embodiments, the at least one disposable sleeve may be configured with a reflective inner surface at an apex, wherein the reflective inner surface may be shaped to create a collimated beam of light and to produce beam divergence.

In some embodiments, the diffusion device may include a diffusion tip configured to illuminate the treatment site, and the least one disposable sleeve may include a disposable tube made from polypropylene. In some embodiments, the at last one disposable sleeve may include a microchip configured to identify the disposable sleeve or the diffusion device. In some embodiments, the at least one disposable sleeve may contain a portion with a shape of an aspheric collimating lens.

In some embodiments, the at least one piece in the medical procedure kit may be at least one cotton roll or swab. In some embodiments, the at least one piece in the medical procedure kit may be a plurality of rolls of variable absorbance material having different shapes or sizes. In some embodiments, the plurality of rolls of variable absorbance material may be texturized cotton.

In some embodiments, the medical procedure kit may further include at least one applicator configured for application of the at least one topical antibiotic. In some embodiments, the at least one applicator may be a non-absorbing applicator having a size and shape configured for medicinal application to the nasal passage. In some embodiments, the at least one topical antibiotic in the medical procedure kit may include mupirocin.

In some embodiments the optical radiation generation device may include at least one diode laser configured to produce dual wavelength NIR at 870 and 930 nanometers (nm). In some embodiments, the delivery assembly may be configured to generate a “flat-top” energy profile.

### BRIEF DESCRIPTION OF THE DRAWINGS

The accompanying drawings, which are incorporated herein and constitute part of this specification, illustrate exemplary embodiments, and together with the general description given above and the detailed description given below, serve to explain the features of the various embodiments.

FIG. 1 illustrates an example of a therapeutic light treatment system suitable for use in various embodiments,

FIGS. 2A-2C are diagrams if cleaning tools that may be include in a medical procedure kit according to various embodiments.

FIGS. 3A-3C are examples of the shapes and configurations for applicators that may be include in a medical procedure kit according to various embodiments.

FIG. 4 is a perspective view of an example diffusion tip in the therapeutic light treatment system shown in FIG. 1.

FIG. 5 is a diagram a disposable sleeve for a laser tip configured with a light diffusing surface on an inside surface for use as an optical diffuser tip of a therapeutic light delivery system according to various embodiments.

FIG. 6 is a diagram a disposable sleeve configured with an aspheric collimating lens on the optical diffuser tip of the therapeutic light delivery system according to various embodiments.

FIG. 7 is s a process flow diagram illustrating a method for treating or preventing microbial infection in the nares according to various embodiments.

### DETAILED DESCRIPTION

Various embodiments include a kit of equipment to assist in effective photobiologic decolonization of S. aureus in the nasal cavity.

In particular, various embodiments provide a kit suitable for use with a therapeutic system for microbial reduction, such as those described in U.S. Pat. No. 8,983,257, which is hereby incorporated by reference. The therapeutic system may be used for a treatment site in a patient's nasal cavity, and may include an optical radiation generation device configured and arranged to generate near-infrared (NIR) therapeutic light; a controller operatively connected to the optical radiation generation device for controlling dosage of the therapeutic light transmitted to the treatment site at a dosimetry sufficient to produce photo damage in the biological contaminant; a delivery assembly including an optical fiber which directs the therapeutic light to be transmitted to the treatment site; and a diffuser tip adapted to receive the therapeutic light from the delivery assembly and diffuse the therapeutic light to illuminate at least a portion of the treatment site with a prescribed illumination pattern.

In various embodiments, the kit may include one or more disposable sleeves configured to be used on the diffusion tip. The one or more disposable sleeves may be transparent to the NIR wavelengths generated by the optical radiation generation device.

In various embodiments, the kit may include one or more tubes or containers of a topical antibiotic such as Mupirocin.

In the last twenty-five years, different groups have studied and explored many of the different elements thought responsible for long-term staphylococcal nasal carriage. Many of these studies have placed an emphasis and focus on bacterial binding to mucosal cells and secreted mucin. While it is recognized that S. aureus exists in secreted mucin and on mucosal cells in the nares, this mucosal area posterior to the vestibulum nasi is not agreed to be the primary ecological niche for this important commensal bacteria.

To achieve the highest possible efficacy for infection prevention in any nasal decolonization therapy, the correct etiologic factors first need to be identified in the accurate ecological niche. Topical application of Mupirocin is currently the primary instrument of nasal decolonization therapy, but such treatment has not been historically 100% successful. Without wishing to be bound by a particular theory, the inventor believes that, the primary the ecological niche inside the nares for S. aueius colonization in the nose may be the vibrissae (specialized hair follicles) of the vestibulum nasi. Further, the inventor believes that failure of nasal decoonisation therapy may occur due to failure of the topical antimicrobial to fully penetrate the ecological niche of the deep recesses of the vebrissae, as well as bacterial resistance to topical antimicrobials and bacterial biofim formation to prevent antimicrobial penetration into the bacteria.

Past errors in nasal decolonization efforts may be found by analyzing a series of accumulated facts and data that a priori point conclusively to vestibulum nasi and the vibrissae as the area of greatest nasal staphylococcal colonization. Specifically, the vestibulum nasi is the distinctive band of skin, containing the vibrissae and sebatious glands, that act as the ecological niche for Staphylococcal colonization, to a far greater extent than the deeper mucosal tissue posterior to the vestibulum nasi.

The “vibrissae” and sebaceous glands, first described from 1886-1906 (in the skin that makes up the most anterior section of the nares), have in most cases overlooked by the modern medical community in staphylococcal decolonization studies. Yet these specialized hair follicles are of the utmost importance when attempting to achieve successful decolonization of the human nostril. Also overlooked when discussing staphylococcal nasal carriage is the presence of vellus hair follicles and sebaceous glands surrounding the vestibulum nasi, which can also harbor S. aureus.

First Years of Data Regarding Bacteria in Human the Hair Follicles

With respect to the earliest mentions in medical literature of micro-organisms of infected hair follicles, in 1889, Park described a self-experiment conducted by German physician Max Bockhart in which Bockhart introduced a minute portion of “cultures of aureus and albus” on the skin of his own forearm, about the size of a silver dollar. It was reported that after “rubbing in” this inoculum, that within 24 hours pustules had formed with most of the pustules being perforated by hairs. After a week, only two pustules remained, but they then developed into large blisters, and boils that caused recurrence of the pustules in the same area for up to three months. This infection of the hair follicle is known as Bockhart impetigo, which is a superficial follicular pustular eruption involving hairy areas. This self-experiment by Bockhart appeared to support that the bacteria could in fact colonize and infect human hair follicles.

A self-experimentation of another German physician and pathologist, Curt Schimmelbusch, was described by Park in 1891. Schimmelbusch had rubbed into unbroken skin pure cultures of staphylococcus pyogenes aureus, and experienced pustules that broke out on the skin. Schimmelbusch then excised an area of the infected skin, prepared and sectioned the biopsy, and further gram stained the skin sample with contrast. Finally, upon microscopic examination, Schimmelbusch found no injury to the skin tissues, but that the staphylococcus had followed the hair shaft down into its follicle, and there proliferated, and reported that the infection of the tissues proceeded from the infected follicle.

In 1895, Thomson and Hewlett made a series of observations about bacteria in the vestibulum nasi, while experimenting at King's College Hospital in London. The results were that the interior of the great majority of normal nasal cavities is perfectly aseptic but that thee vibrissae (nasal hairs) lining the vestibules of the nares were generally swarming with bacteria Thomson and Hewlett described that in all bacterioscopic investigations of the nasal fossae, in all researches as to the action of the nasal mucus, a clear distinction must be made between the vestibule of the nose and the proper mucous cavity. That is, the former is lined with skin, is furnished with hairs and with sudoriferous and sebaceous glands, and is not part of the nose cavity but only leads to it. Thomson and Hewlett also described that in the mucus and debris deposited among the vibrissae of healthy subjects, microorganisms are never absent, but instead are abundant.

Broader Consensus Regarding the Microbiology of the Vestiulum Nasi

In 1913, Allen described an extensive protocol to clean the nasal vestibule and vibrissae with soap, sterile water and alcohol, along with then utilizing a sterile speculum to avoid the vibrissae when attempting to culture desired areas of the nose, posterior to the vestibulum nasi. This protocol was created to avoid cross contamination with bacterial species from the vibrissae, and sebaceous glands of the vestibule. In 1922, Phillips described the etiology of nasal furunculosis as caused by pyogenic staphylococcus, which gain access to the subcutaneous tissues of the nares through hair follicles (of the vibrissae) or the sudoriparous (sweat) glands. In 1926, Herzig reported on the efficacy of a combination of the antiseptics Gentian violet and acriflavine, for the treatment of external bacterial infections, such as nasal furunculosis.

By 1925, experimental and observational data strongly pointed to the vestibulum nasi, and particularly the vibrissae as being most heavily colonized with S. aureus. These data presented a prevailing and early signal as to where future treatments for nasal decolonization should be focused: (1) the skin of the vestibulum nasi; (2) the vibrissae of the vestibulum nasi; and (3) and the sudoriparous glands of the vestibulum nasi.

In 1929, Fleming presented data concerning the in vitro eradication of staphylococcal colonies harvested from the human nose, in his seminal paper describing penicillin.

In 1932, the Norwegian dermatologist Danbolt showed that 35 patients with recurring nasal furunculosis had staphylococci with the same biochemical properties in the nose and in the infected hair follicle lesions in other areas. Danbolt suggested that the nasal staphylococcal colonization was accountable for the recurrent skin infection, representing autoinfection (i.e., infection from a source within the patient itself).

In 1934, Davis conducted an early experiment concerning adequate masking of operating room staff during surgery, and found that for each individual in the operating suite, more staphylococci could be cultured from exhalation of the staff when only the mouth was masked, compared to the mouth and nose being masked. In 1939, Gillespie, et al. demonstrated that there was an association between nasal carriage and skin carriage of S. aureus. In 1939, Devenish and Miles reported the positive finding of S. aureus in the nose of two surgeons, one of which was reported to have far greater rates of surgical site infections. One year later, there was a report of topical administration of aluminum chloride by Veach, who treated a group of patients with chronic Staphylococcal furunculosis in the nose with a 25% aqueous solution, producing a successful treatment outcome for all.

In 1941, Delafield tested sulphathiazole and a sample of “newly purified” penicillin in snuff compounds, to attempt to eliminate nasal carriage of staphylococcI, and found a great reduction in nasal colonies that then returned to normal after the snuff therapy was discontinued. Also in 1941, Thomas presented evidence that the nasal carriage of staphylococci could be reduced (not eliminated) by patients utilizing a “chemotherapeutic” snuff at an isolation hospital for diphtheria. In 1944, Taylor et al. reported the successful use of penicillin spray in the treatment of impetigo and furunculosis. Then in 1944, Gissane et al. discussing the importance of wound antisepsis for healing, clearly pointed to the nose, as a source from which a wound may become infected with Staphylococcus aureus.

In 1945, Lovell reported that the source of the resident flora in microscopically stained skin sections was the sebaceous glands.

By 1945 it was the opinion of the vast majority of physicians and scientists that the hair follicles and sebaceous glands of the vestibulum nasi are the largest source of infective colonies of S. aureus. Physicians of that time had determined that S. aureus could not only self-infect at distant sites in a single person, but also could also infect others. The further significant philosophy put forward in this time frame was that: An antimicrobial in the nose could be used as a a prophylactic to diminish the number of existing pathogens in the nasal cavities, and an antimicrobial in the nose would have to be continued for a long time to effect any permanent cure.

Early Treatment Studies of Nasal Staphylococcus Carriage

In 1947, Hobbs et al. reported that patients receiving penicillin cream to both skin lesions and the nose of patients with sycosis barbae, harbored the identical type of bacteria, and that and all but one patient healed to satisfaction with penicillin cream. In 1948, Moss et al. deduced that skin carriage of S. aureus is dependent on nasal carriage in patients with normal skin. When Moss employed local penicillin treatment (to the nose), but not systemic treatment, the treatment reduced nasal S. aureus carriage from 97% to 37% and simultaneously reduced skin carriage from 57% to 38% after five days of treatment.

In 1950, Evans et al. showed that the bacterial count of the skin varies tremendously from individual to individual, and, from time to time on the same individual, noting that the colony count was much higher in areas well supplied with sebaceous glands, in further confirmation of the conclusions of Lovell in 1945.

In 1955, J. C. Gould conducted a study of multiple different topical antibiotic formulations in the nares, to attempt to prevent the infectivity of the staphylococcus carriers, by suppressing their staphylococci carriage. Gould showed that it was necessary to employ concentrations up to 10,000 units per gram to the surface of the nares to penetrate into the sebaceous glands, and he continued the treatment for 14 days. He found that there was a decline in colony numbers in all patients during the time of antibiotic application and that seven days after the start of treatment no colonies could be cultured from 96 out of 124 carriers.

Gould further stated that topical application of 1% antibiotic cream was effective in suppressing Staph pyogenes in the human nose for an appreciably longer time than the cream was applied. Gould found that it is unlikely that the organism ceased to colonize the skin glands of the nares but rather was not present on the surface at the time of swabbing, nut concluded that the infectivity was still reduced since the staphylococcus was likely not being passed to other sites on the body, nor disseminated into the air and dust.

In 1959, Williams et al. tested for post-operative wound sepsis in patients with staphylococcal nasal carriage, finding that the incidence of post-operative staphylococcal wound sepsis was 2% in 342 patients who were never nasal carriers of staphylococci and 7.1% in the 380 who carried at some time, and that in about half the cases the sepsis was due to a staphylococcus of the same type as was found in the nose.

In 1961 and 1963, Varga and White found a significant relationship between staphylococci in both the nasal carriage and contaminated air samples, and that that nasal administration of oxacillin not only decreased nasal colonization, but also decreased the aerial colonies of S. aureus.

In 1972 Selwyn and Ellis experimented on full thickness skin biopsies from a series of “sudden death” cadavers that had been stored under refrigeration for less than 24 hours, finding. \mean bacterial counts ranging from 4,400/cm² on the breast to 400,000/cm² in the axillae.

Selwyn and Ellis concluded that the organisms were presumably protected from disinfection by lipids, especially at the mouths of follicles, or by overlying portions of the stratum corneum. They found no direct evidence that bacteria are normally found in deep layers of the epidermis outside pilosebaceous units.

Therefore, by the beginning of the 1970s there was little doubt that: (a) The hidden source of the majority resident flora that re-colonize the skin is the hair follicle and sebaceous glands, (b) Skin carriage of S. aureus is heavily influenced by nasal carriage, (c) 1% (10,000 units per gram) antibiotic topicals appear to be of a high enough concentration to penetrate into the sebaceous glands and hair follicles of the nares to lower colony counts, (d) Post-operative staphylococcal wound sepsis is higher in patients with staphylococcal nasal carriage, and a person with nasal carriage can shed substantial amounts of bacteria into the air, and (e) nasal administration of topical antibiotics not only decreased nasal colonization, but also decreased the aerial colonies of S. aureus, that can transfer to other sites on the same person, and other people.

Critical information garnered from the original Mupirocin studies against S. aureus

In 1976, Sutherland et al. presented data showing that pseudomonic acid carried antibacterial activity against gram-positive bacteria such as S. aureus, but not substantial activity against gram-negative enterobacteriacea or gram-positive enterococci. Sutherland also showed no cross-resistance with other antibiotics, and suggested a novel mechanism of action.

In 1978, Hughes and Mellows reported that pseudomonic acid was effective against S. aureus at concentrations of 0.05-0.5 μg/ml, and that the mutant inhibitory concentration (MIC) against S. aureus is 0.05 μg/ml and is bacteriostatic. They also reported that cells treated with pseudomonic acid within the MIC range, once transferred to fresh media, will spontaneously recover after several hours, and that higher concentrations are necessary for a bactericidal effect. They further described inhibition of protein synthesis by reversibly binding to isoleucyl transfer-RNA synthetase, as the novel mechanism of action for pseudomonic acid.

The first human study results for a pseudomonic acid preparation were published in August 1983 by Wuite et al. who studied 46 patients referred to his clinic with pyogenic skin infections. The patients were given one or more tubes of a water miscible cream containing 2% pseudomonic acid (20,000 units per gram) to apply to the infected area three times per day for five days, and returned to the clinic at day seven. In 43 of the 46 patients, the infection was reported as completely cleared.

In 1985, the FDA approved a “Nasal Formulation” of mupirocin in Paraffin, indicated for “the eradication of nasal colonization with methicillin-resistant Staphylococcus aureus (MRSA) in adult and pediatric patients” that had no contraindications for use on nasal or mucosal tissues. In 1986, Casewell and Hill conducted the first moderately sized controlled trial, with the newly approved formulation of 2% mupirocin in white soft paraffin. Thirty-six subjects that tested positive for stable nasal carriage with S. aureus were recruited into the study, and 18 of them were given mupirocin and 18 give the identical base without mupirocin. All participants were instructed to apply an amount of paraffin “the size of a match head” four times per day, and to squeeze their nose between finger and thumb after each application to ensure even distribution. Culture swabs were examined after one, four, eight and 20 applications.

Immediately after each swab that resulted in the elimination of nasal carriage, extra swabs were also taken from the nose, perineum, axillae and wrists of all subjects. Follow-up nasal swabs were taken from all subjects two days after the final application, again and once per week for five weeks. Casewell and Hill reported that all mupirocin treated patients had S. aureus carriage eliminated from their nares. None of the patients were shown to eliminate nasal carriage in the control vehicle arm. Three weeks after the treatment phase, three patients regained S. aureus in the nares, without an increase in the MIC to mupirocin. Also, in 1986, the Hospital Infection Society and the British Society for Antimicrobial Chemotherapy recommended treatment of nasal carriers of MRSA with mupirocin in paraffin applied to the anterior nares three times/day (t.i.d.) for at least five days.

Mupirocin (Paraffin) Nasal Decolonization Studies

In 1988, Hill, et al., reported on a hospital outbreak of MRSA where 40 patients and 32 hospital staff that tested as stable nasal carriers of MRSA received topical application of 2% mupirocin in paraffin to their anterior nares for t.i.d. for five days. Hill reported that nasal carriage was eliminated in all treated patients and staff, usually within the first 48 hours of treatment. Four patients recolonized within two weeks after mupirocin therapy Immediately after the mupirocin therapy was completed, the number of patients with MRSA isolated from wrists fell from 16 to three.

Also in 1989, M. Bulanda et al. treated 69 volunteers with either persistent, intermittent or transient S. aureus carriage in the anterior nares with mupirocin in paraffin t.i.d. for five days. The mupirocin treatment eradicated S. aureus from 67 of the 69 participants when tested four days after the last mupirocin dose. At two weeks post therapy, approximately 40% of the patients had recolonized with S. aureus in the nose.

In 1991, D. R. Regan et al. treated 68 patients with stable S. aureus carriage, in a double-blind, placebo-controlled randomized trial. Participants received either mupirocin in paraffin, or placebo intra-nasally twice daily (b.i.d.) for five days. Regan measured cultures of the hands and nares at baseline and 72 hours after therapy. They reported that the proportion of hand cultures positive for S. aureus in the mupirocin group after therapy was significantly lower than in the placebo group (2.9% compared with 57.6%). Regan concluded that when applied intra-nasally twice daily for five days, mupirocin in paraffin has a corresponding effect on hand carriage at 72 hours after therapy.

Van Rijen et al. performed a systematic review of Staphylococcus aureus infections in surgical patients with nasal carriage between 2002 and 2006 and collected data on a large series of studies meeting criteria for an S. aureus nasal eradication component, utilizing mupirocin nasal for a b.i.d.-five day regimen. Van Rijen et al. highlighted that nasal carriage is only eliminated in approximately 80% of patients treated with mupirocin and 30% in those treated with placebo, when following the b.i.d. regimen.

In 2010, Bode et al. reported on a randomized, double-blind, placebo-controlled, multicenter trial that treated MSSA patients with mupirocin nasal ointment and chlorhexidine soap, to assess any reduction in hospital-associated MSSA infection. Bode et al. reported that 1,270 nasal swabs from 1,251 patients were positive for MSSA, that 917 of the patients were enrolled in the intention-to-treat analysis, of whom 808 (88.1%) underwent a surgical procedure, and that all of the S. aureus strains were susceptible to methicillin and mupirocin. The rate of S. aureus infection was 3.4% in the mupirocin-chlorhexidine group compared with 7.7% in the placebo group. The effect of mupirocin-chlorhexidine treatment was most pronounced for deep surgical-site infections. Therefore, Bode et al. concluded that the number of surgical-site MSSA infections acquired in the hospital can be reduced by decolonizing of nasal carriers of S. aureus on admission.

Overall, the results from clinical studies of nasal decolonization studies using various mupirocin treatments are summarized in the table below:

| | Application | Number | | Elimination | |
|---|---|---|---|---|---|
| Year | Per Day | of Days | | after trial | Recolonization |
| 1986 | 4X per Day | 5 | Days | 100% | 16% at 3 weeks |
| 1989 | 3X per Day | 5 | Days | 100% | 6% at 2 weeks |
| 1989 | 3X per Day | 5 | Days | 97% | 40% at 2 weeks |
| 1991 | 2X-3X per day | 3-8 | days | 97% | Unknown |
| 1992 | 2X per day | 5 | days | 91% | Unknown |
| 1992 | 2X per day | 5 | days | 74% | 22% at 4 weeks |
| 1994 | 2X per day | 5 | days | 91% | 26% at 4 weeks |
| 1995 | 2X per day | 5 | days | 86.7% | 43% at 4 weeks |
| 2002 | 2X per day | 5 | days | 82% | Unknown |
| 2002 | 2X per day | 5 | days | 83% | Unknown |
| 2006 | 2X per day | 5 | days | 81% | Unknown |

Thus, without wishing to be bound to a particular theory, the data demonstrate that various resistance mechanisms have likely come to fore over the thirty years that mupirocin has been FDA approved used in the nose to attempt nasal decolonization.

Current regimens for mupirocin decolonization in the United Kingdom generally include application of mupirocin 2% two to three times per day, without specifying the number of days. Current regimens for mupirocin decolonization in the United States generally include nasal application of mupirocin 2% two times per day for five days.

Of great importance to any future therapy is that there has never been a nasal decolonization study that has not seen recolonization of some measure from the hair follicles and sebaceous glands within a two-to-three week period.

Modern hair follicle studies, and methods for increasing mupirocin penetration into the hair follicle and sebaceous glands.

In 1984, Kearney et al., reported results on a human cadaver skin biopsy study and essentially confirmed that Propionibacteria and Staphylococci are found heterogeneously in high densities in all parts of the follicular canal, and concluded that complete skin disinfection would require the penetration of disinfectant throughout the whole pilosebaceous canal including for the use of topical antibiotics in disease states such as acne vulgaris.

In 2001 and 2005, it was determined that sebum production follows a defined circadian pattern, in a 24-hour cycle. Human Sebum production peaks at 1:00 PM, and has troughs between 11:00 PM and 6:00 AM.

In 2006, Lademann et al., studying nano-particle penetration into hair follicles, suggested that the follicle acts as a geared pump for nanoparticles, if their size is comparable with the thickness of the hair shaft dandruff. Lademann et al. concluded that the reservoir of the hair follicles is a long-term reservoir, because depletion can only occur during slow processes—that is, by penetration into deeper tissue layers or by flowing out with the sebum production.

In 2007, Lademann presented in vitro data that showed that particle penetration increased into hair follicles when massage was applied, and stated that this encourages hair movement, which in vivo occurs physiologically.

In 2010, Broeke-Smits et al. performed the first series of experiments attempting to “define” the ecological niche in the nose for S. aureus to the Vestibulum nasi. Positive S. aureus cultures in nine out of 37 nose swabs, and in the microscopy slides, S. aureus was found in the Vestibulum nasi only. No bacteria were detected in the ciliated mucosa covering the major part of the nose or in its associated serous glands. Broeke-Smits et al reported that: (1) the majority of the bacteria were found within the cornified layer of the stratified squamous epithelium and in the associated keratin and mucous debris within the vestibulum, (2) in six out of nine culture-positive noses the bacteria were also detected in the outer portion of the hair follicle shafts, and (3) two out of six hair follicle-positive noses, bacteria were detected in deeper parts of the hair follicle.

In 2012, Matard reported on the first evidence of the presence of bacterial biofilms in the infra infundibular (deeper portion) of human scalp hair follicles, (in both folliculitis decalvans patients and in healthy subjects), utilizing field emission scanning electron microscopy and laser confocal scanning microscopy.

In 2013, Alexeyev introduced a new table codifying bacterial population sampling methods that included (a) swab, (b) scrape (c) cyanoacrylate and (d) biopsy. Alexeyev found that the “swab method” only identifies bacterial colonies at the level of the superficial stratum corneum. In 2014, Jans et al. presented further evidence in infected folliculitis patients of “large biofilm-like macro colonies in the deep part of the hair follicle. Also in 2014, Ulmer et al. stated that the hair follicles could be used as a reservoir for topically applied substances, that “non-particular” topicals could be detected for up to four days in a follicle after delivery, and that liposomes could also represent an effective long-term drug carrier system within the follicular pathway. Ulmer et al. concluded that the effectiveness of skin antisepsis can be improved by standardized mechanically assisted application and prolonged exposure.

Based on the above findings, a number of overall concussions may be drawn used with respect to the penetration of mupirocin into the hair follicle. Such conclusions include that: (1) temperature can influence the degree of drug deposition in the hair follicle, (2) drug depletion in the hair follicle is a slow processes and can either go deeper by penetration into deeper tissue layers or by flowing out of the follicle with the sebum production, (3) penetration of substances into the follicle can be increased when massage is applied, (4) as much as 25% of the cutaneous bacterial population can be sequestered within the hair follicles, (5) bacterial biofilms have been observed in the deeper portion of the human hair follicle, (6) the “swab method” for bacterial culture only identifies bacterial colonies at the level of the superficial stratum corneum, (7) large biofilm-like macro colonies have been observed in the deep part of the hair follicle, and (8) topicals may be detected for up to four days in a follicle after delivery.

There exists a significant difference between mupirocin resistance on the part of bacteria and therapeutic mupirocin failure (in the absence of genetic mupirocin resistance) to clear bacterial strains from the nares of actual patients in need of mupirocin success.

A number of important factors that serve to result in mupirocin failure have been cataloged, even when the MIC of mupirocin is well within therapeutic range, without expressing bacterial resistance mechanisms. In summary, these factors include that: (1) bacteria seek out places in the nares where traditional mupirocin therapy and paraffin vehicles cannot easily penetrate, examples of which include the hair follicles, sebaceous glands, and the keratinocytes of the nares, (2) the existence of bacterial biofilm in the hair follicles and sebaceous glands also serves as a defensive barrier to mupirocin insult, (3) normal secretion of sebum from the sebaceous glands, inhibits mupirocin penetration into the hair follicles and sebaceous glands, (4) there is a circadian component to sebum secretion, such that mupirocin therapy will most likely be more effective when administered after 1:00 PM following laser therapy and curettage, and (5) mupirocin therapy administered t.i.d. (three times per day) shows statistically significantly better outcomes than when administered b.i.d. (twice per day).

Various embodiments provide a medical procedure kit for improving therapeutic light delivery systems used for a nasal passage of a subject. Such therapeutic light delivery systems may include those that use near infrared optical radiation in selected energies and dosimetries (i.e., “near infrared microbial elimination system” or “NIMELS”) to cause a depolarization of membranes within the irradiated field, altering the absolute value of the membrane potential (ΔΨ) of the irradiated cells. Devices that use NIMELS therapy may include those that enhance minimum inhibitory concentration (MIC) of the antimicrobial agent necessary to attenuate or eliminate microbial related pathology. Detailed descriptions of various example therapeutic light delivery systems that may be used in the various embodiments are provided in U.S. Pat. No. 8,983,257, the entire content of which is incorporated by reference herein.

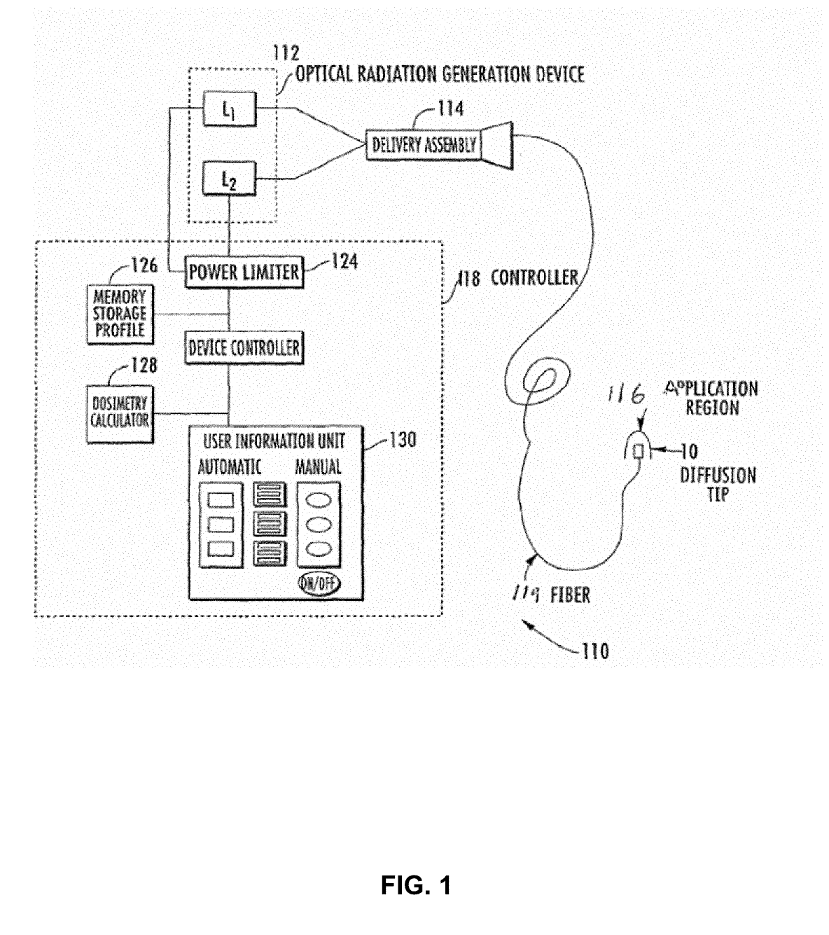

FIG. 1 illustrates an example of a therapeutic light delivery system that may be used in various embodiments. The therapeutic system 110 includes an optical radiation generation device 112, a delivery assembly 114, an application region 116, and a controller 118.

The optical radiation generation device (source) may include one or more suitable lasers, L1 and L2. A suitable laser may be selected based on a degree of coherence. The therapeutic system can include at least one diode laser configured and arranged to produce an output in the near infrared region. Suitable diode lasers may include semiconductor materials for producing radiation in desired wavelength ranges (e.g., dual wavelength radiation at 850 nm-900 nm and 905 nm-945 nm). Suitable diode laser configurations can include cleave-coupled, distributed feedback, distributed Bragg reflector, vertical cavity surface emitting lasers (VCSELS), etc.

The delivery assembly 114 may generate a “flat-top” energy profile for uniform distribution of energy over large areas. For example, a diffuser tip 10, may be included which diffuses treatment light with a uniform cylindrical energy profile in an application region 116 (e.g. a nasal cavity as described in the example above). As noted, the optical radiation generation device 112 can include one or more lasers, e.g., laser oscillators L1 and L2. One laser oscillator can be configured to emit optical radiation in a first wavelength range of 850 nm to 900 nm, and the other laser oscillator can be configured to emit radiation in a second wavelength range of 905 nm to 945 nm. In certain examples, one laser oscillator is configured to emit radiation in a first wavelength range of 865 nm to 875 nm, and the other laser oscillator 28 is configured to emit radiation in a second wavelength range of 925 nm to 935 nm. The geometry or configuration of the individual laser oscillators may be selected as desired, and the selection may be based on the intensity distributions produced by a particular oscillator geometry or configuration.

With continued reference to FIG. 1, the delivery assembly 114 may include an elongated flexible optical fiber 118 adapted for delivery of the dual wavelength radiation from the oscillators 26 and 28 to diffuser tip 10 to illuminate the application region 116. The delivery assembly 114 may have different formats (e.g., including safety features to prevent thermal damage) based on the application requirements. For example, in one form, the delivery assembly 114 or a portion thereof (e.g. tip 10) may be constructed with a size and with a shape for inserting into a patient's body. In alternate forms, the delivery assembly 114 may be constructed with a conical shape for emitting radiation in a diverging-conical mariner to apply the radiation to a relatively large area. Hollow waveguides may be used for the delivery assembly 114. Other size and shapes of the delivery assembly 114 may also be employed based on the requirements of the application site. The delivery assembly 114 can be configured for free space or free beam application of the optical radiation, e.g., making use of available transmission through tissue at NIMELS wavelengths described herein. For example, at 930 nm (and to a similar degree, 870 nm), the applied optical radiation can penetrate patient tissue by up to 1 cm or more. Such systems may be particularly well suited for use with in vivo medical devices as described herein.

The controller 118 includes a power limiter 124 connected to the laser oscillators L1 and L2 for controlling the dosage of the radiation transmitted through the application region 116, such that the time integral of the power density of the transmitted radiation per unit area is below a predetermined threshold, which is set up to prevent damages to the healthy tissue at the application site. The controller 118 may further include a memory 126 for storing treatment information of patients. The stored information of a particular patient may include, but not limited to, dosage of radiation, (for example, including which wavelength, power density, treatment time, skin pigmentation parameters, etc.) and application site information (for example, including type of treatment site (lesion, cancer, etc.), size, depth, etc.).

The memory 126 may also be used to store information of different types of diseases and the treatment profile, for example, the pattern of the radiation and the dosage of the radiation, associated with a particular type of disease. The controller 118 may further include a dosimetry calculator 128 to calculate the dosage needed for a particular patient based on the application type and other application site information input into the controller by a physician. In one form, the controller 118 further includes an imaging system for imaging the application site. The imaging system gathers application site information based on the images of the application site and transfers the gathered information to the dosimetry calculator 128 for dosage calculation. A physician also can manually calculate and input information gathered from the images to the controller 118.

As shown in FIG. 1, the controller may further include a control panel 130 through which, a physician can control the therapeutic system manually. The therapeutic system 10 also can be controlled by a computer, which has a control platform, for example, a WINDOWS™ based platform. The parameters such as pulse intensity, pulse width, pulse repetition rate of the optical radiation can be controlled through both the computer and the control panel 30.

In various embodiments, a medical procedure kit may be configured for use with the therapeutic light delivery system. An embodiment medical procedure kit may include, for example, at least one piece that can be used to remove any remaining debris and softened sebum from the openings to the hair follicles and sebaceous glands, and to open up the follicles and sebaceous gland so that there is greater access for a topical antimicrobial. For example, a kit may include at least one cotton roll and/or swab. Some kits may include cotton rolls of many different shapes and sizes depending on the anatomy of the subject. Some kits may include rolls of a variable absorbance material for cleaning and removing debris and sebum from the nares, which may also be of different sizes and shapes. In various embodiments, at least one specifically shaped and/or textured cotton or variable absorbance roll may be designed for use in the nares. In various embodiments the kit may also contain a tube or tubes of a topical antibiotic, or multiple different antibiotics, configured for easy access and dispensability into the nose and/or nares.

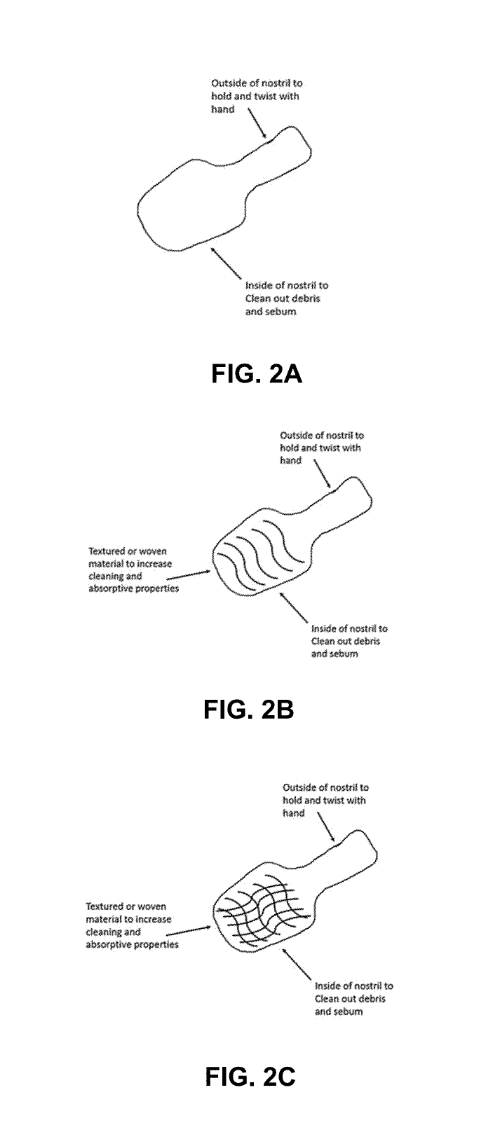

Examples of the shapes and configurations of cotton or other rolls that may be included in an embodiment medical procedure kit are shown in FIGS. 2A-2C.

An embodiment kit may also include at least one piece that can be used for topical application of an antimicrobial agent, such as a cotton applicator. Some kits may include at least one non-absorbing applicator for dispensing topical medicine in the nares. The cotton applicator(s) and/or non-absorbing applicator(s) may be of different sizes and shapes, and may be specifically shaped and textured for enhance topical medicinal application and penetration into the hair follicles and sebaceous glands in the nares.

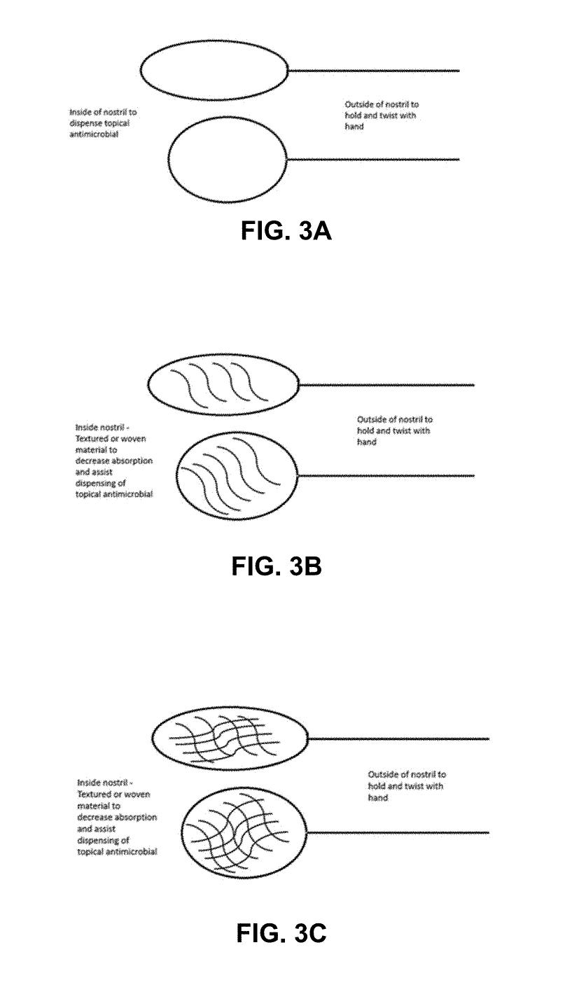

Examples of the shapes and configurations for applicators that may be include in an embodiment medical procedure kit are shown in FIGS. 3A-3C.

Various embodiments also include a medical procedure kit comprising one or more specifically shaped disposable sleeves essentially transparent to the near infrared energy from the diffuser of an optical nasal diffusion device specifically designed for light application in the nares. Specifically, the optical nasal diffusion device may be a diffusion tip (e.g., tip 10) employed by a therapeutic light delivery system (e.g., treatment system 110) to diffuse therapeutic treatment light delivered from a therapeutic light source by an optical fiber.

The diffusion tip operates to provide a desired illumination profile (i.e. emitted intensity profile) at the application region. For example, where treatment light is applied to the nares, a substantially uniform cylindrical illumination profile is desirable. In some systems, a diffusion tip may be used to direct treatment light to other areas such as tissue spaces (e.g. the periodontal pocket or within a joint e.g. in an orthopedic surgical procedure), interfaces between body tissue and other surfaces (e.g. the surface of an implantable medical device), over a wide area such as a dermal surface, etc.).

Specifically, the diffusion tip may be an optically transmissive, light diffusing, fiber tip assembly having an entrance aperture through a proximal reflector, a radiation-scattering, transmissive material surrounding an enclosed void (e.g. a cylindrical cavity), and a distal reflective surface. As radiation propagates through the fiber tip, a portion of the radiation is scattered in a cylindrical (or partly cylindrical) pattern along the distal portion of the fiber tip. Radiation which is not scattered during this initial pass through the tip is reflected by at least one surface of the assembly and returned through the tip. During this second pass, the remaining radiation, (or a portion of the returning radiation), is scattered and emitted from the proximal portion of the tube. Multiple additional reflections off of the proximal and distal reflectors provide further homogenization of the intensity profile. Preferably the scattering medium has a prescribed inner diameter. This inner diameter of the scattering material is designed such that the interaction with this material and the multiple reflections off of the cavity reflectors interact to provide a substantially proscribed axial distribution of laser radiation over the length of the tip apparatus.

Various diffusion tip configurations may be employed in treatment system used in various embodiments. For example, some diffusion tips substantially uniform energy distribution to a major portion of the exposure area, while some provide for constructing and implementing circumferential and/or sideways emitting diffusing tip assemblies for optical fibers to direct laser radiation in a radially outward pattern relative to the fiber's longitudinal axis. As used herein, the term “optical fiber” is intended to encompass optically transmissive waveguides of various shapes and sizes.

Some diffusion tip configurations are intended for a higher aspect ratio of length to diameter. Typical aspect ratios for prior art diffusing tip technologies may be from 20 to 1 and higher. (e.g. 1 mm diameter and 20 mm length). Some diffusion tip configurations allow for producing diffusing tip assemblies with aspect ratios of about 10 or less, about 1 or less, or about 0.1 or less.

The diffusion tip may be provided as an assembly used for diffusing radiation from an optical fiber. The tip assembly may include a light transmissive, tubular housing, alignable with, and adapted to receive, the distal end of the fiber and serve as a diffusive scattering medium for light that has been emitted by the optical fiber. The assembly further includes a reflective cavity formed by reflectors on each side of the diffusive tube, such that the light is scattered by the tube on its first pass through the tube and is emitted outward to the illumination site. The un-scattered portion of the illumination is reflected back to further interact with the scattering tube. This second pass illumination is then scattered outward by the scattering tube to complement the light emitted on the first pass to produce the desired illumination profile. Additional 2nd, 3rd and 4th reflections with subsequent scattering from the diffusing tube can be added to produce additional homogeneity of the emitted axial energy profiles.

The reflective surfaces of the apparatus can also be modified to effect non-planar forms. Reflective structures are disclosed which control the spatial distribution of the light emitted from the tip. These techniques and structures permit, for example, an evenly distributed orthogonal projection of the radiation.

The diameter of the tubular scattering material and/or the length of the diffusion tip can be controlled such that the diffusion of the radiation during the initial and reflected paths are complementary. By proper choice of such parameters, the cumulative energy profile, or fluence, along at least a portion of the fiber tip can be rendered uniform. The term “substantially uniform” is commonly used in the field of phototherapy to describe light diffusers that possess a uniformity of about +/−15% or less of the average intensity of light emitted from the diffusive tip assembly. Thus, the diffusion tip (e.g., tip 10) provides a mechanism for substantially uniform cylindrical illumination of biological structures and other illumination applications.

Some diffusion tips may be used to apply therapeutic light at NIMELS dosimetry and wavelengths without exhibiting heating to temperatures which are unwanted or intolerable at the treatment site (i.e. temperatures that would cause substantial thermal damage at the site, or discomfort to a patient undergoing treatment). For example, the diffusion tip may absorb about 20% or less of the therapeutic light delivered from a therapeutic source at NIMELS dosimetry and wavelengths. The diffusion tip may be operated to deliver therapeutic light at NIMELS dosimetry and wavelengths for treatment times on the order tens of seconds or on the order of minutes or more while remaining at an operating temperature of 110° F. degrees or less, or 100° F. degrees or less.

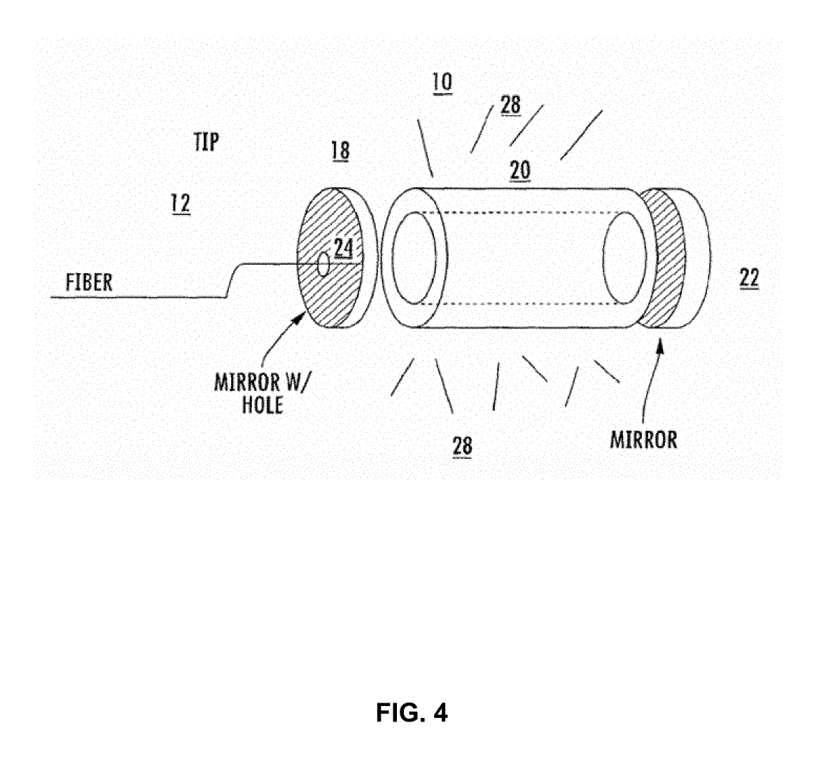

FIG. 4 illustrates an example of the diffusion tip 10, which is an assembly that includes an optical fiber 12 having a light transmissive core 14, a cladding 16, a proximal first mirror 18, a diffusing tube 20, and a distal second mirror 22. The end face of fiber 12 is inserted through an aperture 24 in the first mirror 18.

Each medical procedure kit in various embodiments may also include at least one disposable sleeve for the diffusion tip of the therapeutic light delivery system. The disposable sleeve may be made of an optically-transmissive medical grade plastic. At least one disposable sleeve in each kit may be transparent to one or multiple NIR wavelengths, such as enabling NIR wavelengths of 870 and 930 nanometers (nm) to pass through largely unimpeded. In some embodiments, a disposable sleeve may be configured as transparent to at least some wavelengths in order to prevent heat build-up.

Example materials that may be used to make disposable sleeves for the medical procedure kit, and their properties and/or applications, include:

| Material | Properties/Typical Applications |
|---|---|
| Calcium | Low Absorption, High Refractive Index Homogeneity |
| Fluoride | Used in Spectroscopy, Semiconductor Processing, Cooled |
| (CaF₂) | Thermal Imaging |
| Fused | Low CTE and Excellent Transmission in IR |
| Silica (FS) | Used in Interferometry, Laser Instrumentation, |
| | Spectroscopy |
| Germanium | High n_{d}, High Knoop Hardness, Excellent MWIR |
| (Ge) | to FIR Transmission |
| | Used in Thermal Imaging, Rugged IR Imaging |
| Magnesium | High CTE, Low Index of Refraction, Good Transmission |
| Fluoride | from Visible to MWIR |
| (MgF₂) | Used in Windows, Lenses, and Polarizers that Do Not |
| | Require Anti-Reflection Coatings |
| N-BK7 | Low-Cost Material, Works Well in Visible and NIR |
| | Applications |
| | Used in Machine Vision, Microscopy, Industrial |
| | Applications |
| Potassium | Good Resistance to Mechanical Shock, Water Soluble, |
| Bromide | Broad Transmission Range |
| (KBr) | Used in FTIR Spectroscopy |
| Sapphire | Very Durable and Good Transmission in IR |
| | Used in IR Laser Systems, Spectroscopy, and Rugged |
| | Environmental Equipment |
| Silicon | Low Cost and Lightweight |
| (Si) | Used in Spectroscopy, MWIR Laser Systems, THz Imaging |
| Sodium | Water Soluble, Low Cost, Excellent Transmission from |
| Chloride | 250 nm to 16 μm, Sensitive to Thermal Shock |
| (NaCl) | Used in FTIR spectroscopy |
| Zinc | Low Absorption, High Resistance to Thermal Shock |
| Selenide | CO₂ Laser Systems and Thermal Imaging |
| (ZnSe) | |
| Zinc Sulfide | Excellent Transmission in Both Visible and IR, Harder and |
| (ZnS) | More Chemically Resistant than ZnSe |
| | Used in Thermal Imaging |

In some embodiments, at least one disposable sleeve provided in the kit may be configured with a parabolic or spherical reflective inner surface at its apex. In some embodiments, the parabolic or spherical reflective inner surface may reflect excess NIR light traveling away from the treatment site back to the nasal passage.

In some embodiments, the apex of a disposable sleeve provided in the kit may be far enough away from the light source as to cause a diffuse spread of the excess light back towards the nares, as opposed to a straight collimated beam.

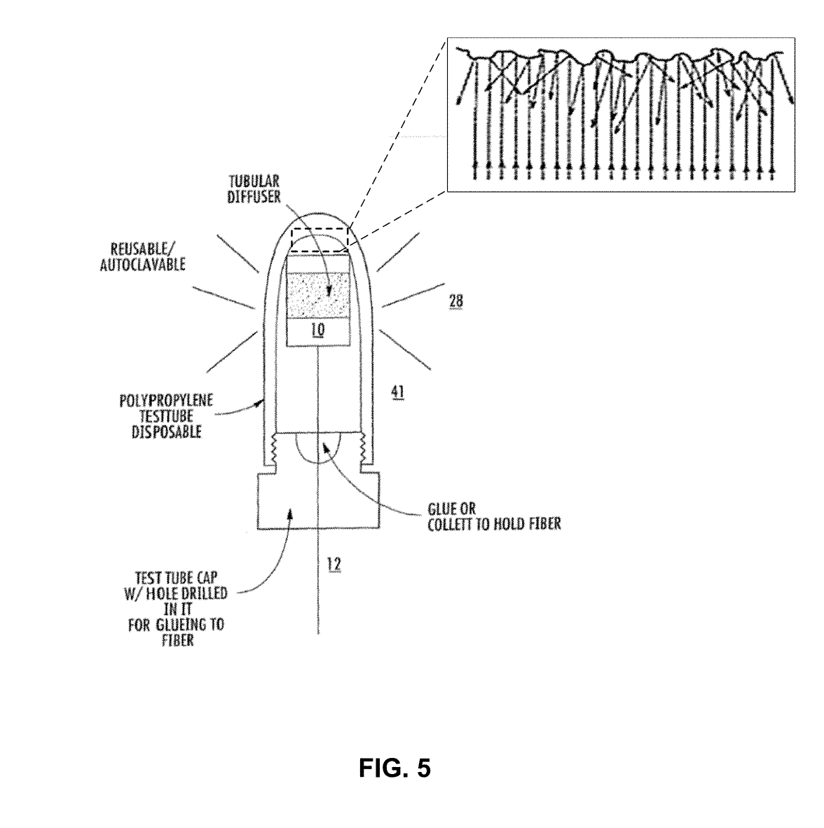

The reflective surface may be a “pebbled” reflective material that has the effect of producing micro-scattering of the light. An example of a disposable sleeve configured with a “pebbled” (i.e., irregular) reflective inner surface at the apex on the optical diffuser tip of the therapeutic light delivery system (e.g., 110 in FIG. 1) is shown in FIG. 5. Specifically, a diffusion tip may be enclosed by a disposable, sterile, test tube sized appropriately for the diffusion tip assembly. A preferred disposable tube is made from Polypropylene, due to its high transmission of visible and near infrared light, non-shattering nature and ability to withstand high temperatures. Alternate materials may include polycarbonate or Pyrex glass. In some embodiments, the diffusion tip 10 may be autoclavable and also reusable.

In another embodiment, the reflective surface may be a soft “matte” reflective material.

In other embodiments, the parabolic or spherical reflective geometry may be partially relaxed, resulting in an incompletely collimated beam of light that also produces beam divergence.

In some embodiments, the disposable sleeve may include a microchip as an identification mechanism (e.g., encryption mechanism, authentication mechanism, single use mechanism, etc). The microchip may be positioned to interface with the therapeutic output laser dispersion head (e.g., diffusion tip or other diffusion device). In other words, in this example, the microchip and the therapeutic output dispersion head are interconnected (e.g., electrically, physically, etc.).

In some embodiments, the identification mechanism may enable the sleeve to only be utilized once. In other words, in this example, the sleeve is a one-time use medical apparatus that is disposed of after use in the nares. The microchip can include a destruction mechanism that self-destructs after a specified time period of use (e.g., ten seconds, twenty seconds, etc.). In other words, in this example, the destruction mechanism destroys the identification mechanism and the destruction of the identification mechanism prevents the sleeve from being re-used since the therapeutic output head cannot verify the identity of the sleeve.

In other embodiments, the microchip includes an encryption mechanism that enables authentication of the sleeve with the therapeutic output head. The therapeutic output head and/or the therapeutic device may query the encryption mechanism to determine the identity of the sleeve. The encryption mechanism may respond with an authentication response (e.g., key, signature, security token, etc.). The therapeutic output head and/or the therapeutic device may process the authentication response to verify that the sleeve is valid and/or other information associated with the sleeve (e.g., that the sleeve can operate with the therapeutic output head, therapeutic parameters of the sleeve, the serial number of the sleeve, the manufacturer of the sleeve). If the sleeve is not validated, the therapeutic output head and/or the therapeutic device may automatically de-activate until a validated sleeve is connected to the therapeutic output head. The authentication mechanism for the disposable sleeve may advantageously protect patients by ensuring that the sleeves are not re-used, i.e., the safety of the patient is increased.

In some embodiments, the pieces may be packaged for use with the therapeutic output system for photo biologic nasal decontamination. For example, the items may be packaged as either large and/or small. In some embodiments, the disposable sleeve may include a connection mechanism. Every piece in the kit may be provided in ready-to-use condition in a packaging arrangement.

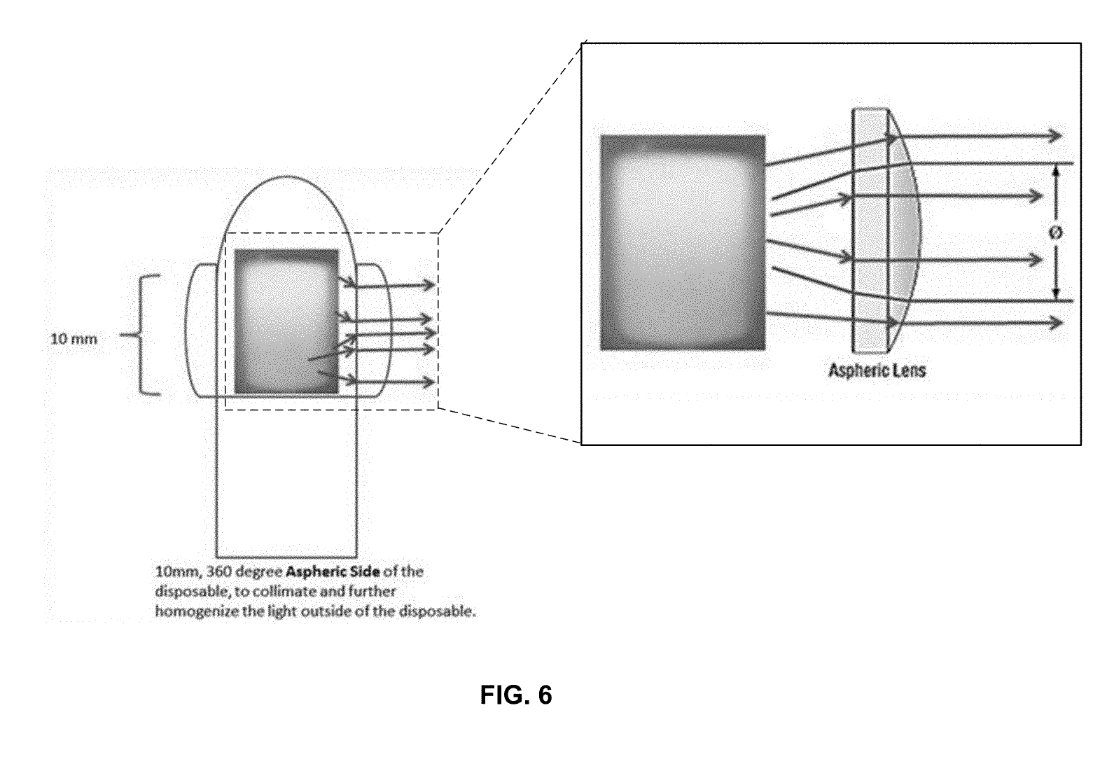

In some embodiments, the at least one disposable sleeve may include a portion that is shaped like an aspheric collimating lens. In particular, the portion of a disposable sleeve that will be positioned inside the patient's nostril and surrounding the diffusing tip may be aspheric. The therapeutic light delivery system (e.g., therapeutic system 110) may have a diffuser tip 10 that is 10 mm long and may extend slightly more than 10 mm into a patient's nose. To reduce the angle of light existing the diffuser tip 10, natural properties of an aspheric collimating lens may be incorporated into the 10 mm length of the disposable sleeve around the diffuser. An example of such aspheric collimating lens properties is shown in FIG. 5. An example of a disposable sleeve configured with such aspheric collimating lens properties on the optical diffuser tip of the therapeutic light delivery system (as shown in FIG. 6).

In some embodiments, a disposable sleeve may incorporate both the beam divergence configuration of the apex shown in FIG. 5 and the aspheric collimating lens configuration around the diffuser tip shown in FIG. 6.

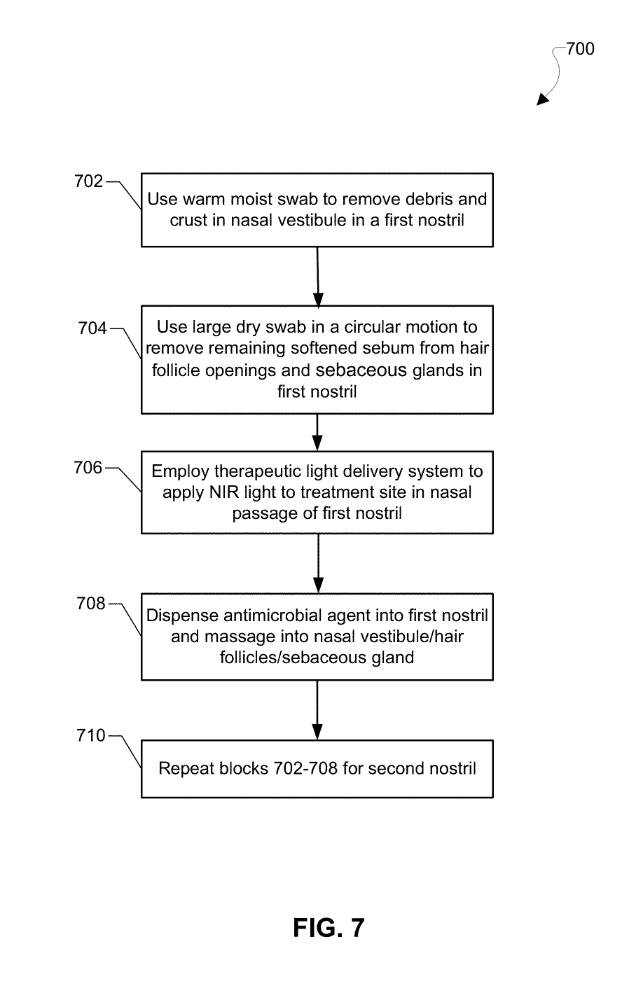

When all of these factors are taken into account and combined with the potentiation effect of the laser therapy, in various embodiments, improvements described above may be applied in new treatment protocols for photobiologic eradication of S. aureus and MRSA in the nares, as shown in FIG. 7. In various embodiments, the new treatment protocol 700 may be used t.i.d. for a duration of 5-7 days. In block 702 First, a large warm moist swab may be used to remove any debris and crust in the nasal vestibule in a first nostril. In various embodiments, the swab can be made of multiple different fabrics and have many different shapes and sizes depending on the anatomy of the patient. In step 704, a large dry swab may be used in a circular motion to remove any remaining softened sebum from the openings to the hair follicles and sebaceous glands in the first nostril, in order to open up the follicles and sebaceous glands prior to mupirocin nasal delivery. In various embodiments, this swab may be made of multiple different fabrics and of many different shapes and sizes depending on the anatomy of the patient.

In block 706, a therapeutic light delivery system (e.g., therapeutic system 110) may be employed to apply NIR light to a treatment site in the nasal passage of the first nostril. In block 708, an antimicrobial agent (e.g., mupirocin nasal) may be dispensed into the first nostril and massaged into the nasal vestibule, as well as the nasal hair follicles and sebaceous gland on the outside of the nasal vestibule. In some embodiments, such massaging may be performed by, for 60 seconds, repeatedly squeezing the first nostril with the thumb and forefinger. In block 710, the steps in blocks 702-708 may be repeated for the second nostril.

The preceding description of the disclosed embodiments is provided to enable any person skilled in the art to make or use the claims. Various modifications to these embodiments will be readily apparent to those skilled in the art, and the generic principles defined herein may be applied to other embodiments without departing from the scope of the claims. Thus, the present disclosure is not intended to be limited to the embodiments shown herein but is to be accorded the widest scope consistent with the following claims and the principles and novel features disclosed herein.

What is claimed is:

1. A therapeutic light delivery system for performing photo-biologic nasal decolonization of bacteria within a nasal passage of a subject, comprising:
an optical radiation generation device;
a controller;
a delivery assembly comprising:
an optical fiber adapted for transmitting near-infrared (NIR) radiation to a treatment site within the nasal passage; and
a diffusion device; and
a medical procedure kit comprising:
at least one piece for use in removing debris and softening sebum from the openings to hair follicles and sebaceous glands in the nasal passage;
at least one disposable sleeve for the diffusion device; and
at least one topical antibiotic for dispensing into the nasal passage.

2. The system of claim 1, wherein the at least one disposable sleeve is made of an optically-transmissive medical grade plastic that is transparent to one or multiple NIR wavelengths.

3. The system of claim 1, wherein the at least one disposable sleeve is configured with a parabolic or spherical reflective inner surface at an apex.

4. The system of claim 3, wherein the parabolic or spherical reflective inner surface is configured to produce micro-scattering of light.

5. The system of claim 3, wherein the parabolic or spherical reflective inner surface is configured to reflect excess NIR light traveling away from the treatment site back to the nasal passage.

6. The system of claim 5, wherein the apex of the at least one disposable sleeve is positioned such that a diffuse spread of the excess light back towards the nasal passage is produced.

7. The system of claim 1, wherein the at least one disposable sleeve is configured with a reflective inner surface at an apex, wherein the reflective inner surface is shaped to create a collimated beam of light and to produce beam divergence.

8. The system of claim 1, wherein:
the diffusion device comprises a diffusion tip configured to illuminate the treatment site; and
the least one disposable sleeve comprises a disposable tube made from polypropylene.

9. The system of claim 1, wherein the at last one disposable sleeve comprises a microchip configured to identify the disposable sleeve or the diffusion device.

10. The system of claim 1, wherein the at least one disposable sleeve contains a portion with a shape of an aspheric collimating lens

11. The system of claim 1, wherein the at least one piece comprises at least one cotton roll or swab.

12. The system of claim 1, wherein the at least one piece comprises a plurality of rolls of variable absorbance material having different shapes or sizes.

13. The system of claim 12, wherein the plurality of rolls of variable absorbance material comprise texturized cotton.

14. The system of claim 1, wherein the medical procedure kit further comprises at least one applicator configured for application of the at least one topical antibiotic.

15. The system of claim 14, wherein the at least one applicator comprises a non-absorbing applicator having a size and shape configured for medicinal application to the nasal passage.

16. The system of claim 1, wherein the at least one topical antibiotic comprises mupirocin.

17. The system of claim 1, wherein the optical radiation generation device comprises at least one diode laser configured to produce dual wavelength NIR at 870 and 930 nanometers (nm).

18. The system of claim 1, wherein the delivery assembly is configured to generate a “flat-top” energy profile.
